(19) United States
(12) Patent Application Publication
(10) Pub. No.: US 2026/0249273 A1
(43) Pub. Date: Aug. 27, 2026

(54) CATALYST FOR PREPARATION OF LINEAR ALPHA OLEFIN, METHOD FOR PREPARING SAME AND METHOD FOR PREPARING LINEAR ALPHA OLEFIN USING SAME

(71) Applicant: IUCF-HYU (INDUSTRY-UNIVERSITY COOPERATION FOUNDATION HANYANG UNIVERSITY), Seoul (KR)

(72) Inventors: Jeong Gil SEO, Seoul (KR); Dae Ho HONG, Seoul (KR); Mahlet Nigus GEBRESILLASE, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/650,518

(22) Filed: Apr. 17, 2026

Related U.S. Application Data
(63) Continuation of application No. PCT/KR2024/096333, filed on Oct. 11, 2024.

(30) Foreign Application Priority Data
Oct. 17, 2023 (KR) 10-2023-0138627

(51) Int. Cl. B01J 23/10; B01J 21/04; B01J 35/30; B01J 37/02; B01J 37/08; C07C 1/24

(52) U.S. Cl. CPC B01J 23/10; B01J 21/04; B01J 35/394; B01J 37/0201; B01J 37/0236; B01J 37/088; C07C 1/24

(57) ABSTRACT

The present invention relates to a catalyst capable of improving conversion rate and selectivity in preparing a linear alpha-olefin through a dehydration reaction of a primary alcohol, a method for preparing the same, and a method for preparing a linear alpha-olefin using the same. A catalyst having optimized acid sites and base sites can be provided through an interaction between cerium oxide and aluminum oxide, and by applying the same to a dehydration reaction of a primary alcohol, selectivity and conversion rate for a linear alpha-olefin can be improved. In addition, the yield of a linear alpha-olefin can be improved by controlling conditions such as a supported amount of cerium of the catalyst and a reaction temperature, and linear alpha-olefins can be prepared in an environmentally friendly and economical manner by exhibiting an effect of improving selectivity even for biomass-derived alcohols having 5 or more carbon atoms.

### CROSS-REFERENCE TO RELATED APPLICATIONS

This application is a continuation of International Application No. PCT/KR2024/096333 filed on Oct. 11, 2024, which claims priority to Korean Patent Application No. 10-2023-0138627 filed on Oct. 17, 2023, the entire contents of which are herein incorporated by reference.

### TECHNICAL FIELD

The present invention relates to a catalyst for preparing a linear alpha-olefin, a method for preparing the same, and a method for preparing a linear alpha-olefin using the same, and more particularly, to a catalyst capable of improving conversion rate and selectivity in preparing a linear alpha-olefin through a dehydration reaction of a primary alcohol, a method for preparing the same, and a method for preparing a linear alpha-olefin using the same.

### BACKGROUND ART

Linear alpha-olefins (LAOs) are a general term for linear hydrocarbons having one double bond at the alpha position of a molecular structure, and are used as intermediates for synthesizing various products such as (co) monomers of polymers, lubricants, plasticizers, and surfactants.

Known techniques for synthesizing linear alpha-olefins include oligomerization of ethylene, catalytic cracking of naphtha, and dehydrogenation of paraffin. For example, Korean Patent Publication No. 10-2012-0050963 relates to a method for preparing a linear alpha-olefin by oligomerization of ethylene, and describes a technique of oligomerizing ethylene using a catalyst, followed by injecting an additive and reacting the same. However, this technique has disadvantages in that the selectivity for linear alpha-olefins is low, requiring an additional separation process, and most of the materials used are petroleum-based, making the process not environmentally friendly.

Accordingly, a technique for preparing linear alpha-olefins using a dehydration reaction of alcohols has been attracting attention as an alternative technique. The use of a dehydration reaction of alcohols has an advantage in that linear alpha-olefins can be prepared in an environmentally friendly manner. For example, Korean Patent Registration No. 10-2306324 relates to a technique for preparing alpha-olefins using a dehydration reaction of a primary alcohol, and describes a technique of subjecting a primary alcohol to a dehydration reaction using a catalyst in which Group 6 transition metal particles are supported on a composite metal oxide of magnesium aluminate and hydrotalcite as a support.

However, this technique has a disadvantage in that there is a limitation in improving the selectivity for linear alpha-olefins when applied to alcohols having 5 or more carbon atoms, such as 1-octanol. In addition, other studies utilizing the dehydration reaction of alcohols are mostly directed to alcohols having a small number of carbon atoms, and studies applied to biomass-derived alcohols having 5 or more carbon atoms have not been actively conducted.

Therefore, there is a need for the development of a technique capable of obtaining linear alpha-olefins in high yield in an environmentally friendly manner by improving the dehydration reaction conversion rate and selectivity for linear alpha-olefins using a primary alcohol, while also exhibiting such effects of improving the selectivity and yield of linear alpha-olefins even when applied to alcohols having 5 or more carbon atoms.

### SUMMARY

### Technical Problem

An object of the present invention is to provide a catalyst applicable to a dehydration reaction of a primary alcohol to improve selectivity for a linear alpha-olefin.

Another object of the present invention is to provide a method for preparing the catalyst.

Still another object of the present invention is to provide a method for preparing a linear alpha-olefin in high yield using the catalyst.

### Technical Solution

To achieve the above objects, the present invention provides a catalyst for preparing a linear alpha-olefin, comprising cerium oxide (CeO₂) supported on aluminum oxide (Al₂O₃).

In the catalyst of the present invention, a supported amount of cerium may be 3 to 30% by weight based on a total weight of the catalyst.

In the present invention, an acid site of the catalyst may be 0.80 to 1.20 mmol/g.

In the catalyst of the present invention, an acid site of a strong acid may be 30 to 40% of a total acid site.

In the present invention, a base site of the catalyst may be 0.80 to 1.20 mmol/g.

In the catalyst of the present invention, a base site of a strong base may be 35 to 40% of a total base site.

The present invention may also provide a method for preparing the catalyst for preparing a linear alpha-olefin.

The method for preparing a catalyst for preparing a linear alpha-olefin of the present invention may comprise: preparing a mixed solution comprising a cerium salt and aluminum oxide; and preparing a catalyst comprising cerium oxide (CeO₂) supported on aluminum oxide (Al₂O₃) by drying and calcining the mixed solution.

In the present invention, the cerium salt may be at least one selected from the group consisting of cerium nitrate, cerium ammonium nitrate, cerium ammonium sulfate, cerium acetate, cerium sulfate, cerium carbonate, cerium hydroxide, cerium chloride, cerium bromide, cerium fluoride, and cerium iodide.

In the present invention, the solvent of the mixed solution may be at least one selected from the group consisting of water (distilled water), acetone, alcohol, ethyl acetate, hexane, heptane, toluene, dichloromethane, tetrahydrofuran, acetonitrile, dimethylacetamide, dimethylformamide, dimethyl sulfoxide, dimethyl oxalate, and methylpyrrolidone.

In the present invention, the calcining may be performed by increasing a temperature at a rate of 2 to 8° C./min under a condition of 350 to 600° C.

The present invention may also provide a method for preparing a linear alpha-olefin using the catalyst.

The linear alpha-olefin may be prepared by subjecting a primary alcohol to a dehydration reaction in the presence of a catalyst comprising cerium oxide (CeO₂) supported on aluminum oxide (Al₂O₃).

In the present invention, the primary alcohol may be at least one linear primary alcohol selected from the group consisting of 1-propanol, 1-butanol, 1-pentanol, 1-hexanol, 1-heptanol, 1-octanol, 1-nonanol, and 1-decanol.

In the present invention, the dehydration reaction may be performed at a temperature of higher than 200° C. and 400° C. or lower.

In the present invention, the method for preparing a linear alpha-olefin may comprise: loading and activating the catalyst in a reactor; and obtaining a linear alpha-olefin by introducing a primary alcohol into the reactor and subjecting the primary alcohol to a dehydration reaction.

In the present invention, the activating of the catalyst may be performed at 300 to 700° C.

### Advantageous Effects

According to the present invention, a catalyst having optimized acid sites and base sites can be provided through an interaction between cerium oxide and aluminum oxide, and by applying the same to a dehydration reaction of a primary alcohol, selectivity and conversion rate for a linear alpha-olefin can be improved. In addition, in the present invention, the yield of a linear alpha-olefin can be improved by controlling conditions such as a supported amount of cerium of the catalyst and a reaction temperature, and linear alpha-olefins can be prepared in an environmentally friendly and economical manner by exhibiting an effect of improving selectivity even for alcohols having 5 or more carbon atoms.

### DESCRIPTION OF DRAWINGS

FIG. 1 is a graph showing conversion rate of a dehydration reaction of 1-hexanol and yield of 1-hexene according to a supported amount of cerium of a catalyst in one embodiment of the present invention.

FIG. 2 shows results of measuring acid sites of a CeO₂/Al₂O₃ catalyst using ammonia temperature-programmed desorption (NH₃-TPD) according to one embodiment of the present invention.

FIG. 3 shows results of measuring base sites of a CeO₂/Al₂O₃ catalyst using carbon dioxide temperature-programmed desorption (CO₂-TPD) according to one embodiment of the present invention.

### DETAILED DESCRIPTION

Hereinafter, specific embodiments of the present invention will be described in more detail. Unless otherwise defined, all technical and scientific terms used herein have the same meanings as commonly understood by those skilled in the art to which the present invention pertains. In general, the nomenclature used herein is well-known and commonly used in the art.

Linear alpha-olefins (LAOs) are linear hydrocarbons having one double bond at the alpha position of a molecular structure. Linear alpha-olefins are mainly produced through dehydrogenation of paraffin, oligomerization of ethylene, Fischer-Tropsch process, and the like, all of which are based on petrochemical materials. Accordingly, techniques for preparing linear alpha-olefins have generally had a disadvantage in that they are associated with environmental problems such as depletion of petroleum and acceleration of global warming, problems of raw material supply and demand due to fluctuations in oil prices, and economic problems due to cost fluctuations.

The present invention can solve such problems, and relates to a catalyst capable of preparing a linear alpha-olefin in an environmentally friendly and economical manner using a dehydration reaction of a primary alcohol.

The dehydration reaction of a primary alcohol proceeds at an acid site of a catalyst, and for example, the preparation of an alpha-olefin through a dehydration reaction of 1-hexanol proceeds according to the following Reaction Scheme.

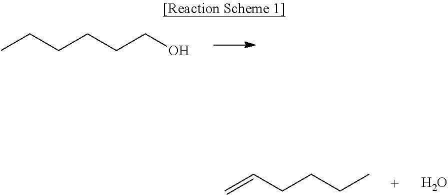

However, since the activation energy for an etherification reaction is thermodynamically lower than that for producing an alpha-olefin through a dehydration reaction of an alcohol, dihexyl ether is prepared at a low temperature as shown in the following Reaction Scheme.

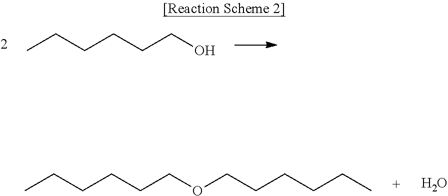

As such, since dialkyl ether is produced through etherification, which is a side reaction of alcohol, at a low temperature during the dehydration reaction of a primary alcohol, high temperature conditions are required for selectively preparing a linear alpha-olefin. However, under high temperature conditions, there is a problem in that isomerization of an alpha-olefin occurs through a strongly acidic catalyst, producing various isomers.

In the present invention, the selectivity and yield of a linear alpha-olefin can be improved by using a catalyst comprising cerium oxide (CeO₂) supported on aluminum oxide (Al₂O₃).

In the present invention, the aluminum oxide used in the catalyst may be γ-aluminum oxide, that is, aluminum oxide having a porous structure formed by dehydrating boehmite at a high temperature.

The catalyst according to the present invention has enhanced acid sites and base sites due to an interaction between cerium and aluminum oxide, and thus exhibits excellent conversion rate and high selectivity for a linear alpha-olefin when used for a dehydration reaction of an alcohol, and can also exhibit excellent performance even when applied to alcohols having 5 or more carbon atoms.

In this regard, in an embodiment of the present invention, dehydration reactions of 1-hexanol were performed using a KCC-1LHPW catalyst, an aluminum oxide catalyst, an aluminum oxide catalyst supported with niobium oxide, a cerium oxide catalyst, and an aluminum oxide catalyst supported with cerium oxide, respectively, and as a result, it was confirmed that an aluminum oxide catalyst supported with cerium oxide exhibited significantly superior selectivity for 1-hexene compared to other catalysts.

In addition, in the present invention, the selectivity and conversion rate for a linear alpha-olefin can be further improved by optimizing the acid sites and base sites of the catalyst through controlling the supported amount of cerium.

In the present invention, the supported amount of cerium in the catalyst may be 3 to 30% by weight, preferably 5 to 25% by weight, specifically 5 to 15% by weight, and more preferably 8 to 12% by weight, based on the total weight of the catalyst.

When the supported amount of cerium is too small, the interaction between cerium oxide and aluminum oxide is not sufficiently exhibited, and when the supported amount of cerium is too large, saturated cerium oxide may agglomerate, causing a problem of decreased dispersibility.

In this regard, in the Examples of the present invention, catalysts were prepared by controlling the supported amount of cerium to 5, 10, 15, and 25% by weight, and dehydration reactions of 1-hexanol were performed using the same, and as a result, it was confirmed that when the supported amount of cerium was 10% by weight, the conversion rate was as high as 99.2% and the selectivity for 1-hexene was excellent at 88.8%, resulting in a high yield of 1-hexene of 88.1%. As such, in the present invention, the yield of a linear alpha-olefin can be increased by improving the conversion rate of a primary alcohol and the selectivity for a linear alpha-olefin by controlling the supported amount of cerium.

In the present invention, a linear alpha-olefin can be obtained in high yield when applied to a dehydration reaction of a primary alcohol due to a change in the strength of acid sites and the basicity of cerium oxide through an interaction between cerium oxide and aluminum oxide.

The dehydration reaction of a primary alcohol proceeds at an acid site of a catalyst, and since a catalyst having too strong acidity causes production of isomers through a double bond shift, it is important to control the acid sites of the catalyst for selective production of an alpha-olefin.

In the present invention, the acid site of the catalyst may be 0.80 to 1.20 mmol/g, and preferably 1.05 to 1.15 mmol/g. In particular, in the catalyst of the present invention, the acid site of aluminum oxide is enhanced to a strong acid by cerium oxide, so that the acid site of the strong acid may be 30% or more, specifically 30 to 40%, and preferably 35 to 40% of the total acid site.

In addition, the base site of the catalyst may be 0.70 to 1.00 mmol/g, and preferably 0.95 to 0.99 mmol/g. In the present invention, the base site is enhanced to a strong base by an interaction between cerium oxide and aluminum oxide, so that the base site of the strong base may be 35% or more, specifically 35 to 40%, and preferably 37 to 40% of the total base site.

As such, in the present invention, the selectivity and conversion rate for a linear alpha-olefin can be significantly improved by applying a catalyst having optimized acid sites and base sites to a dehydration reaction of a primary alcohol.

The present invention may also provide a method for preparing a catalyst supported with cerium oxide on aluminum oxide.

The catalyst of the present invention may be prepared through an impregnation method, and specifically may be prepared through: preparing a mixed solution comprising a cerium salt and aluminum oxide; and forming a catalyst supported with cerium oxide on aluminum oxide by drying and calcining the mixed solution.

The cerium salt is a precursor of cerium oxide, and may be, for example, at least one selected from the group consisting of cerium nitrate, cerium ammonium nitrate, cerium ammonium sulfate, cerium acetate, cerium sulfate, cerium carbonate, cerium hydroxide, cerium chloride, cerium bromide, cerium fluoride, and cerium iodide. The cerium salt may be used in a hydrate form.

The cerium salt and aluminum oxide are mixed in a solution. In this case, the solvent of the solution may be at least one selected from the group consisting of water (distilled water), acetone, alcohol, ethyl acetate, hexane, heptane, toluene, dichloromethane, tetrahydrofuran, acetonitrile, dimethylacetamide, dimethylformamide, dimethyl sulfoxide, dimethyl oxalate, and methylpyrrolidone, and is preferably distilled water.

In the mixed solution, the concentration of aluminum oxide may be 0.1 to 3 g/mL, preferably 0.5 to 2 g/mL, and the concentration of the cerium salt may be appropriately controlled according to the supported amount of cerium of the catalyst to be prepared.

To prepare the mixed solution, the mixed solution may be prepared by diluting a cerium salt in a solvent, adding aluminum oxide thereto, and then mixing the same.

When the mixed solution is prepared, a step of drying the same to obtain a solid sample is performed.

The drying may be performed using known techniques and devices such as an oven, hot air, constant temperature and humidity, and microwave, and may be performed at a temperature of 40 to 100° C., preferably 50 to 80° C., to completely remove the solvent.

After all the solvent is evaporated through the drying process, a catalyst is prepared through a step of calcining the solid sample at a high temperature.

The calcining may be performed at a condition of 350 to 600° C., preferably 400 to 500° C., while increasing the temperature. In this case, the temperature increase rate may be 2 to 8° C./min, preferably 4 to 6° C./min, and the total calcining time may be 2 to 6 hours, preferably 3 to 5 hours.

Through the calcining process, a catalyst in powder form supported with cerium oxide on aluminum oxide can be obtained.

In the catalyst prepared as such, cerium oxide binds to strong Lewis acid sites of aluminum oxide to lower the acid sites of the catalyst, while increasing the base sites of the catalyst through the Lewis acid sites and basicity that cerium oxide itself possesses, thereby enabling selective preparation of a linear alpha-olefin in a dehydration reaction of a primary alcohol.

The present invention may also provide a method for preparing a linear alpha-olefin using the catalyst.

According to the present invention, a linear alpha-olefin can be prepared by subjecting a primary alcohol to a dehydration reaction in the presence of the catalyst. Specifically, the method for preparing a linear alpha-olefin may be performed by: loading and activating the catalyst in a reactor; and introducing a primary alcohol into the reactor and subjecting the primary alcohol to a dehydration reaction.

The activating of the catalyst may be performed at 300 to 700° C., preferably 400 to 600° C., and the catalyst is activated by maintaining the temperature for 1 to 4 hours, followed by introducing a primary alcohol to initiate the reaction.

The primary alcohol used in the reaction may be at least one linear primary alcohol selected from the group consisting of alcohols having 3 to 10 carbon atoms, that is, 1-propanol, 1-butanol, 1-pentanol, 1-hexanol, 1-heptanol, 1-octanol, 1-nonanol, and 1-decanol, and the linear alpha-olefin produced therefrom may be at least one selected from the group consisting of linear alpha-olefins having 3 to 10 carbon atoms, 1-propene, 1-butene, 1-pentene, 1-hexene, 1-heptene, 1-octene, 1-nonene, and 1-decene. In particular, the present invention is preferable in that a reaction with high selectivity and conversion rate for a linear alpha-olefin can be performed even when applied to alcohols having 5 or more carbon atoms.

In the reaction, upon introducing a primary alcohol, the reaction may be proceeded in a manner of introducing the primary alcohol under a nitrogen flow condition of 10 to 1,000 mL/min, for example, 50 to 200 mL/min, and then stabilizing the flow.

In the present invention, the temperature of the dehydration reaction may be controlled to a temperature of higher than 200° C. and 400° C. or lower, and preferably may be controlled to 250° C. or higher and lower than 400° C. When the temperature is low, the etherification reaction of a primary alcohol proceeds and ether is mainly produced, resulting in low selectivity for a linear alpha-olefin. On the other hand, when the temperature is too high, isomerization of an alpha-olefin is promoted, which may cause a problem of decreased selectivity and purity. In this regard, 280 to 380° C. may be more preferable as the temperature of the dehydration reaction.

In this regard, in the Examples of the present invention, dehydration reactions of 1-pentanol, 1-hexanol, and 1-octanol were performed at 150, 200, 250, 300, 350, and 400° C., respectively, and as a result, it was confirmed that in the case of 1-pentanol, the conversion rate and selectivity were excellent at conditions of 300° C. and 350° C., and in particular, the yield of 1-pentene was the highest at 300° C. In addition, in the case of 1-hexanol, the conversion rate was as high as 90% or more at conditions of 300° C., 350° C., and 400° C., and the selectivity for 1-hexene was the most excellent at 88.8% at 350° C., resulting in a high yield of 88.1%, and in the case of 1-octanol, the conversion rate was as high as 90% or more at 250, 300, 350, and 400° C., and the selectivity for 1-octene was the highest at 64.4% at 300° C.

Accordingly, it was confirmed that the temperature range of the dehydration reaction of a primary alcohol using the catalyst of the present invention is preferably 280 to 380° C., and the specific optimal temperature varies depending on the number of carbon atoms of the alcohol, with 280 to 320° C. being most preferable for 1-pentanol and 1-octanol, and 330 to 380° C. being most preferable for 1-hexanol.

As such, in the present invention, a linear alpha-olefin can be prepared in high yield by reacting a primary alcohol in the presence of the catalyst and optimizing the reaction temperature. In addition, the catalyst of the present invention exhibits the above effects even in the dehydration reaction of a primary alcohol having 5 or more carbon atoms, and thus can effectively prepare a linear alpha-olefin using biomass-derived 1-pentanol, 1-hexanol, 1-octanol, and the like. Therefore, the present invention can be effectively used in the development of technology for producing high value-added compounds derived from biomass.

### EXAMPLES

Hereinafter, the present invention will be described in more detail through Examples. However, these Examples represent some experimental methods and configurations for illustratively explaining the present invention, and the scope of the present invention is not limited to these Examples.

### Preparation Example: Preparation of a Catalyst Supported with Cerium Oxide on Aluminum Oxide

A CeO₂/Al₂O₃ catalyst was prepared by an impregnation method using a cerium oxide precursor and aluminum oxide.

Cerium nitrate hexahydrate (Ce(NO₃)₃·6H₂O), a precursor of cerium oxide, was diluted in 10 mL of distilled water to prepare a mixture, and 1 g of gamma-aluminum oxide (γ-Al₂O₃) was added to the well-diluted mixture, followed by stirring for 6 hours. The prepared mixture was placed in an oven and all distilled water was evaporated at 80° C., and then the resulting solid sample was placed in an electric furnace and calcined at 450° C. at a temperature increase rate of 5° C./min for 4 hours.

Using the above method, catalysts having supported amounts of Ce of 5, 10, 15, and 25% by weight, respectively, based on the total weight of the catalyst were prepared, and each catalyst was denoted as nCeO₂/Al₂O₃ (n=mass percent content of Ce). For example, in the case of the Ce 10% catalyst, 0.310 g of cerium nitrate hexahydrate was used, calculated based on the molecular weight of cerium nitrate hexahydrate (434.22 g) and the atomic weight of cerium (140.116 g), and the weight of cerium nitrate hexahydrate used was controlled according to the supported amount of Ce.

### Experimental Example 1: Analysis of Dehydration Reaction Performance of 1-Hexanol According to Catalyst Type

A dehydration reaction of 1-hexanol was performed using the 10CeO₂/Al₂O₃ catalyst prepared according to the method of the Preparation Example, and the conversion rate and selectivity were measured.

The dehydration reaction was conducted in a quartz tube (outer diameter 6 mm, inner diameter 4 mm, length 330 mm) of a continuous catalytic reactor, and the temperature was controlled using a vertical tube electric furnace. Gas flow paths were formed through ¼-inch stainless steel gas lines at the upper and lower ends of the reactor, and an auxiliary path was constructed so that gas could flow to either the reactor or the auxiliary path through valve operation. Heating tape was wrapped tightly around the gas lines, and heat was applied through a slidax to control the temperature of the gas, and a thermocouple was installed on the gas lines to confirm and maintain the temperature of the gas at 200° C.

50 mg of the catalyst was fixed inside the quartz tube using quartz wool, and before the reaction, the reactor temperature was increased to 500° C. over 1 hour under a nitrogen flow of 30 mL and maintained for 2 hours to activate the catalyst. After activation of the catalyst, the temperature of the reactor was adjusted to 350° C. At the same time, the gas flow was switched to the auxiliary path, and 0.3 mL/h of 1-hexanol was injected with a syringe pump under a nitrogen flow of 100 mL/min, and the flow of 1-hexanol was stabilized for 30 minutes. The gas flow was switched from the auxiliary path to the reactor, and the reaction was proceeded under a weight hourly space velocity (WHSV) of 5 h⁻¹, and the start of the reaction was recorded immediately after switching the gas flow from the auxiliary path to the reactor.

The reaction products were analyzed using an online GC-FID (Chrozen GC, YoungIn Chromass) instrument, with a DB-WAX (Agilent J&W, 0.250 mm×0.25 μm×30 m) column for analyzing the alcohol as a reactant and a GS-Gaspro (Agilent J&W, 0.32 mm×60 m) column for analyzing the olefin as a product. Qualitative/quantitative analysis was performed to calculate conversion rate (X), selectivity(S), and yield (Y) using the following equations, and the results are shown in Table 1 below, and for comparison, the same experiments were performed for KCC-1LHPW, Nb₂O₅/Al₂O₃, Al₂O₃, and CeO₂ catalysts, and the results are also shown in Table 1 below. The KCC-1LHPW catalyst refers to a catalyst in which HPW (phosphotungstic acid, H₃PW₁₂O₄₀) is supported on KCC-1 (fibrous nano-silica) as a support (DOI: 10.1007/s11814-021-0768-6).

Conversion rate(X)=[reacted alcohol (mmol/min)/introduced alcohol (mmol/min)]×100(%)

Selectivity(S)=[product (mmol/min)/total products (mmol/min)]×100(%)

Yield(Y)=X×S/100(%)

TABLE 1

| | | | | | | | 1- |
|---|---|---|---|---|---|---|---|
| | | Selectivity (%) | | | | | Hexene |
| | Conversion | 1- | c-2- | t-2- | c-3- | t-3- | yield |
| Catalyst | rate (%) | Hexene | Hexene | Hexene | Hexene | Hexene | (%) |
| KCC- | 83.1 | 35.1 | 23.7 | 19.8 | 3.6 | 17.8 | 29.2 |
| 1LHPW | | | | | | | |
| Nb₂O₅/Al₂O₃ | 90.9 | 40.9 | 21.7 | 18.0 | 3.2 | 16.2 | 37.2 |
| Al₂O₃ | 100 | 7.8 | 24.3 | 32.7 | 6.0 | 29.3 | 7.8 |
| CeO₂/Al₂O₃ | 99.2 | 88.8 | 4.6 | 3.0 | 1.0 | 2.7 | 88.1 |
| CeO₂ | 64.3 | 41.2 | 6.8 | 22.7 | 8.8 | 20.4 | 26.5 |

As a result of performing a dehydration reaction of 1-hexanol using catalysts with various acid properties, catalysts with more acid sites exhibited high conversion rates, but the selectivity for 1-hexene was at a level of 40%. In addition, cerium oxide, which has fewer acid sites and more base sites, exhibited a very low conversion rate, but was confirmed to have a somewhat higher selectivity for 1-hexene compared to other catalysts.

In the case of the aluminum oxide catalyst, a conversion rate of 100% was exhibited, but the isomerization of 1-hexene was activated due to the high acid sites and low basicity of the catalyst, resulting in a significantly decreased selectivity for 1-hexene. However, the catalyst supported with cerium oxide on aluminum oxide exhibited a very high 1-hexanol conversion rate of 99.2%, and furthermore, an excellent selectivity for 1-hexene of 88.8% was obtained, and as a result, 1-hexene could be prepared in a high yield of 88.1%.

### Experimental Example 2: Analysis of Dehydration Reaction Performance of 1-Hexanol According to Supported Amount of Cerium

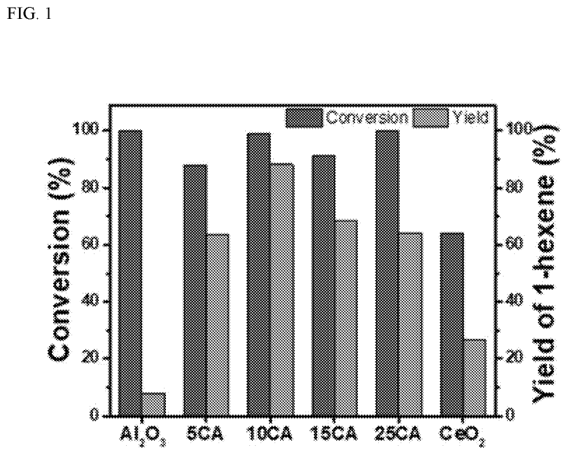

To investigate the effect of cerium oxide in the CeO₂/Al₂O₃ catalyst, catalysts were prepared by controlling the supported amount of cerium to 5, 10, 15, and 25% by weight, respectively. Dehydration reactions of 1-hexanol were performed using each catalyst, and the results of calculating the conversion rate of the reaction, selectivity for olefins, and yield of 1-hexene are shown in FIG. 1 and Table 2 below.

TABLE 2

| | | | | | | | 1- |
|---|---|---|---|---|---|---|---|
| | | Selectivity (%) | | | | | Hexene |
| | Conversion | 1- | c-2- | t-2- | c-3- | t-3- | yield |
| Catalyst | rate (%) | Hexene | Hexene | Hexene | Hexene | Hexene | (%) |
| 5CeO₂/Al₂O₃ | 87.9 | 72.6 | 16.4 | 5.3 | 1.1 | 4.7 | 63.8 |
| 10CeO₂/Al₂O₃ | 99.2 | 88.8 | 4.6 | 3.0 | 1.0 | 2.7 | 88.1 |
| 15CeO₂/Al₂O₃ | 91.4 | 74.7 | 15.9 | 4.5 | 1.0 | 4.0 | 68.3 |
| 25CeO₂/Al₂O₃ | 100 | 64.2 | 21.4 | 6.8 | 1.4 | 6.1 | 64.2 |
| Al₂O₃ | 100 | 7.8 | 24.3 | 32.7 | 6.0 | 29.3 | 7.8 |
| CeO₂ | 64.3 | 41.2 | 6.8 | 22.7 | 8.8 | 20.4 | 26.5 |

According to the above experimental results, when the supported amount of cerium was 5% by weight based on the total weight of the catalyst, the conversion rate and selectivity for 1-hexene were somewhat low, and when the supported amount increased to 25% by weight, the conversion rate increased to 100%, but the selectivity for 1-hexene decreased to 65% or less. However, when the supported amount of cerium was 10% by weight, the conversion rate was as high as 99.2% and the selectivity for 1-hexene was also significantly improved, confirming that the yield of 1-hexene was as high as 88.1%.

Accordingly, it was confirmed that the performance of the CeO₂/Al₂O₃ catalyst was the most excellent when the supported amount of cerium was 10% by weight.

### Experimental Example 3: Analysis of Acid Sites and Base Sites of Catalyst According to Weight Ratio of Cerium Oxide

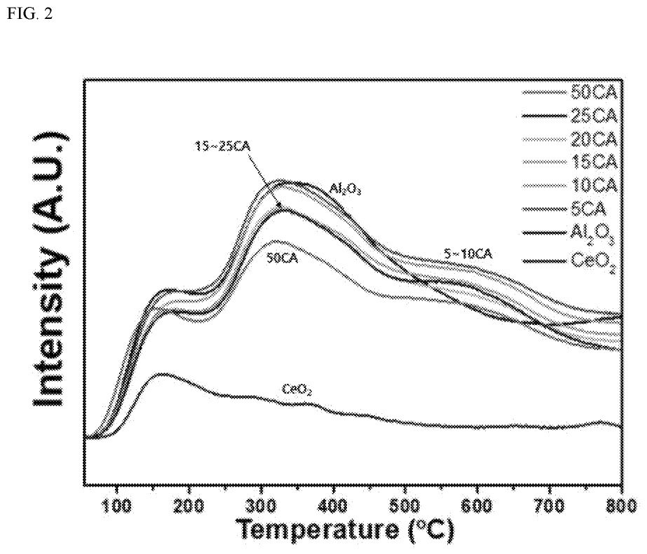

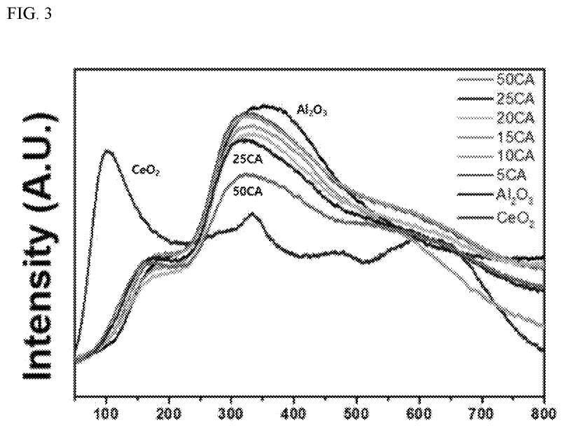

To analyze the reason why the catalyst supported with cerium oxide improves the performance of the alcohol dehydration reaction, ammonia temperature-programmed desorption (NH₃-TPD) and carbon dioxide temperature-programmed desorption (CO₂-TPD) analyses were performed on the Al₂O₃ catalyst, CeO₂ catalyst, and CeO₂/Al₂O₃ catalysts having weight ratios of cerium oxide of 5, 10, 15, and 25%, respectively, and the results are shown in FIG. 2, FIG. 3, and Table 3 below.

TABLE 3

| | Acid site (mmolg⁻¹) | | | | Base site (mmolg⁻¹) | | | |
|---|---|---|---|---|---|---|---|---|
| Catalyst | Weak acid | Medium acid | Strong acid | Total | Weak base | Medium base | Strong base | Total |
| Al₂O₃ | 0.091 | 0.693 | 0.302 | 1.085 | 0.056 | 0.542 | 0.325 | 0.924 |
| CeO₂ | 0.058 | 0.081 | 0.009 | 0.139 | 0.285 | 0.356 | 0.312 | 0.953 |
| 5CA | 0.091 | 0.644 | 0.445 | 1.180 | 0.069 | 0.501 | 0.379 | 0.949 |
| 10CA | 0.081 | 0.605 | 0.417 | 1.103 | 0.066 | 0.530 | 0.364 | 0.960 |
| 15CA | 0.084 | 0.520 | 0.361 | 0.965 | 0.054 | 0.418 | 0.306 | 0.778 |
| 25CA | 0.085 | 0.459 | 0.359 | 0.901 | 0.061 | 0.402 | 0.345 | 0.808 |

As a result, it was confirmed that pure aluminum oxide had more acid sites and fewer base sites than cerium oxide, and in the case of aluminum oxide supported with cerium oxide, the acid sites were partially enhanced from medium acid to strong acid, and the base sites were partially enhanced from medium base to strong base. Accordingly, it was confirmed that the acid sites and base sites were enhanced due to the interaction between cerium and aluminum oxide.

Meanwhile, in the catalyst supported with cerium at 15% by weight or more, saturated cerium oxide agglomerated and the dispersibility decreased, resulting in a decrease in the total acid sites and base sites.

According to the above experimental results, it was confirmed that the acid sites and base sites of the CeO₂/Al₂O₃ catalyst were optimal when the supported amount of cerium was 10% by weight.

### Experimental Example 4: Analysis of Conversion Rate and Selectivity According to Type of Alcohol and Temperature

The catalyst was applied to other primary alcohols, 1-pentanol and 1-octanol, and the performance was confirmed.

The reaction was proceeded in the same manner as in Experimental Example 1, except that the heating temperature using a slidac before catalyst activation was maintained at 170° C. for the reaction of 1-pentanol and at 220° C. for the reaction of 1-octanol.

To confirm the effect of the reaction temperature, dehydration reactions of 1-pentanol, 1-hexanol, and 1-octanol were performed at 50° C. intervals in the temperature range of 150 to 400° C., and the results are shown in Tables 4 to 6 below.

TABLE 4

| | Conver- | 1-Pentene | 1-Pentene | DPE | 1-Pentene |
|---|---|---|---|---|---|
| Temperature | sion rate | selectivity | purity | selectivity | yield |
| (° C.) | (%) | (%) | (%) | (%) | (%) |
| 150 | 53.1 | 1.5 | 17.5 | 91.6 | 0.8 |
| 200 | 61.7 | 3.4 | 36.4 | 90.5 | 2.1 |
| 250 | 85.1 | 41.7 | 83.5 | 50.1 | 35.5 |
| 300 | 100 | 94.7 | 94.7 | 0 | 94.7 |
| 350 | 100 | 89.2 | 89.2 | 0 | 89.2 |
| 400 | 100 | 72.0 | 72.0 | 0 | 72.0 |

TABLE 5

| | Conver- | 1-Hexene | 1-Hexene | DHE | 1-Hexene |
|---|---|---|---|---|---|
| Temperature | sion rate | selectivity | purity | selectivity | yield |
| (° C.) | (%) | (%) | (%) | (%) | (%) |
| 150 | 69.5 | 0 | 0 | 100 | 0 |
| 200 | 61.8 | 4.2 | 86.9 | 95.2 | 2.6 |
| 250 | 87.2 | 45.2 | 65.2 | 30.7 | 39.4 |
| 300 | 94.0 | 43.8 | 43.8 | 0 | 41.2 |
| 350 | 99.2 | 88.8 | 88.8 | 0 | 88.1 |
| 400 | 92.1 | 32.4 | 32.4 | 0 | 29.8 |

TABLE 6

| | Conver- | 1-Octene | 1-Octene | DOE | 1-Octene |
|---|---|---|---|---|---|
| Temperature | sion rate | selectivity | purity | selectivity | yield |
| (° C.) | (%) | (%) | (%) | (%) | (%) |
| 150 | 8.7 | 0 | 0 | 100 | 0 |
| 200 | 57.7 | 34.8 | >99 | 64.9 | 20.1 |
| 250 | 94.2 | 50.2 | 74.9 | 33.0 | 47.3 |
| 300 | 99.8 | 64.4 | 75.1 | 14.2 | 64.3 |
| 350 | 98.2 | 40.6 | 40.6 | 0 | 39.9 |
| 400 | 98.7 | 60.5 | 77.6 | 22.1 | 59.7 |

As a result, at low temperatures of 150 to 200° C., 1-pentanol mainly produced dipentyl ether (DPE), 1-hexanol mainly produced dihexyl ether (DHE), and 1-octanol mainly produced dioctyl ether (DOE), and the selectivity for olefins tended to increase as the temperature increased. Accordingly, it was confirmed that the temperature should be controlled to higher than 200° C., and 250° C. or higher is preferable.

However, when the temperature is raised too high, the isomerization of linear alpha-olefins is promoted or etherification proceeds, resulting in a tendency of decreased selectivity and purity, and thus it was confirmed that the temperature should be controlled to 400° C. or lower, and it is preferable to control the temperature to lower than 400° C.

In the case of 1-pentanol, the conversion rate was 100% and the selectivity was 94.7% at 300° C., exhibiting excellent olefin preparation performance, and 1-hexanol exhibited the highest yield with a conversion rate of 99.2% and a selectivity of 88.8% at 350° C. In addition, 1-octanol exhibited the most excellent yield with a conversion rate of 99.8% and a selectivity of 64.4% at 300° C.

According to the above results, it was found that optimization of the reaction temperature is a very important factor for producing linear alpha-olefins with high selectivity, and that the yield can be further improved by optimally controlling the temperature according to the number of carbon atoms of the alcohol.

Having described specific parts of the present invention in detail above, it will be apparent to those skilled in the art that such specific descriptions are merely preferred embodiments and do not limit the scope of the present invention. Therefore, the substantial scope of the present invention shall be defined by the appended claims and their equivalents.

What is claimed is:

1. A catalyst for preparing a linear alpha-olefin, comprising cerium oxide (CeO₂) supported on aluminum oxide (Al₂O₃).

2. The catalyst for preparing a linear alpha-olefin according to claim 1, wherein a supported amount of cerium is 3 to 30% by weight based on a total weight of the catalyst.

3. The catalyst for preparing a linear alpha-olefin according to claim 1, wherein an acid site of the catalyst is 0.80 to 1.20 mmol/g.

4. The catalyst for preparing a linear alpha-olefin according to claim 3, wherein an acid site of a strong acid in the catalyst is 30 to 40% of a total acid site.

5. The catalyst for preparing a linear alpha-olefin according to claim 1, wherein a base site of the catalyst is 0.80 to 1.20 mmol/g.

6. The catalyst for preparing a linear alpha-olefin according to claim 5, wherein a base site of a strong base in the catalyst is 35 to 40% of a total base site.

7. A method for preparing a catalyst for preparing a linear alpha-olefin, the method comprising:
preparing a mixed solution comprising a cerium salt and aluminum oxide; and
preparing a catalyst comprising cerium oxide (CeO₂) supported on aluminum oxide (Al₂O₃) by drying and calcining the mixed solution.

8. The method for preparing a catalyst for preparing a linear alpha-olefin according to claim 7, wherein the cerium salt is at least one selected from the group consisting of cerium nitrate, cerium ammonium nitrate, cerium ammonium sulfate, cerium acetate, cerium sulfate, cerium carbonate, cerium hydroxide, cerium chloride, cerium bromide, cerium fluoride, and cerium iodide.

9. The method for preparing a catalyst for preparing a linear alpha-olefin according to claim 7, wherein a solvent of the mixed solution is at least one selected from the group consisting of water (distilled water), acetone, alcohol, ethyl acetate, hexane, heptane, toluene, dichloromethane, tetrahydrofuran, acetonitrile, dimethylacetamide, dimethylformamide, dimethyl sulfoxide, dimethyl oxalate, and methylpyrrolidone.

10. The method for preparing a catalyst for preparing a linear alpha-olefin according to claim 7, wherein the calcining is performed by increasing a temperature at a rate of 2 to 8° C./min under a condition of 350 to 600° C.

11. A method for preparing a linear alpha-olefin, comprising obtaining a linear alpha-olefin by subjecting a primary alcohol to a dehydration reaction in the presence of a catalyst comprising cerium oxide (CeO₂) supported on aluminum oxide (Al₂O₃).

12. The method for preparing a linear alpha-olefin according to claim 11, wherein the primary alcohol is at least one linear primary alcohol selected from the group consisting of 1-propanol, 1-butanol, 1-pentanol, 1-hexanol, 1-heptanol, 1-octanol, 1-nonanol, and 1-decanol.

13. The method for preparing a linear alpha-olefin according to claim 11, wherein the dehydration reaction is performed at a temperature of higher than 200° C. and 400° C. or lower.

14. The method for preparing a linear alpha-olefin according to claim 11, wherein the method comprises:
loading and activating the catalyst in a reactor; and
obtaining a linear alpha-olefin by introducing a primary alcohol into the reactor and subjecting the primary alcohol to a dehydration reaction.

15. The method for preparing a linear alpha-olefin according to claim 14, wherein the activating of the catalyst is performed at 300 to 700° C.
